(19) United States
(12) Patent Application Publication
(10) Pub. No.: US 2004/0023880 A1
(43) Pub. Date: Feb. 5, 2004

(54) Methods of inducing formation of functional and organized lymphatic vessels

(76) Inventors: Nicholas W. Gale, Yorktown Heights, NY (US); O. Gavin Thurston, White Plains, NY (US); George D. Yancopoulos, Yorktown Heights, NY (US)

Correspondence Address: REGENERON PHARMACEUTICALS, INC, 777 OLD SAW MILL RIVER ROAD, TARRYTOWN, NY, 10591, US

(21) Appl. No.: 10/427,884

(22) Filed: May 1, 2003

Related U.S. Application Data
(60) Provisional application No. 60/377,924, filed on May 3, 2002.

(51) Int. Cl. A61K 38/18; A61K 48/00; A61K 31/70; A61K 39/395

(52) U.S. Cl. 514/12; 424/146.1; 514/44; 514/23

(57) ABSTRACT

Methods of inducing formation of functional and organized lymphatic vessels are described. Specifically, the methods relate to using Tie2 agonists to induce formation of functional and organized lymphatic vessels. The methods also relate to treating defects, diseases, and disorders characterized by lymphatic vessel malfunction, disorganization, and damage.

This application claims the benefit of U.S. Provisional Application No. 60/377,924, filed May 3, 2002, the contents of which is incorporated herein by reference.

### FIELD OF THE INVENTION

The field of this invention is inducing formation of functional and organized lymphatic vessels. Specifically, the field of this invention relates to using Tie2 agonists to induce formation of functional and organized lymphatic vessels. The field of this invention also relates to methods of treating defects, diseases, and disorders characterized by lymphatic vessel malfunction, disorganization, and damage.

### BACKGROUND

Studies have demonstrated that members of the VEGF family, most likely both VEGF-C and VEGF-D working via VEGFR-3, play critical roles in the development of the lymphatic vasculature (Veikkola, T., et al. (2001) Embo J 20:1223-31). Preliminary analysis of Ang2 knockout (KO) mice suggested an absence of peripheral lymphatics and lymph nodes (Witte, M. H., et al. (2001) Microscopy Research and Techniques 55:122-145).

### BRIEF SUMMARY OF THE INVENTION

Generally, the methods of the invention comprise administering a therapeutically effective amount of pharmaceutical compositions of Tie2 agonists, in an acceptable pharmaceutical carrier, to a subject in need, i.e., a subject afflicted with, for example, lymphedema. In some specific embodiments, the Tie2 agonist is used to treat patients experiencing chylous ascites or ascites associated with liver disease.

In a first aspect, the invention includes a method of promoting functional lymphatic vessel formation comprising administering to a subject a Tie2 agonist such that functional lymphatic formation occurs. In a second related aspect, the invention includes a method of inducing lymphatic vessel maturation comprising administering to a subject a Tie2 agonist such that functional lymphatic maturation occurs. In a third related aspect, the invention is a method of preventing chylous ascites formation comprising administering to a subject a Tie2 agonist such that chylous ascites is not formed. In a fourth aspect, the invention is a method of treating lymphedema comprising administering to a subject a Tie2 agonist such that lymphedema is not formed. In a fifth aspect, the invention is a method of decreasing ascites associated with cirrhosis of the liver comprising administering to subject a Tie2 agonist such that ascites associated with cirrhosis of the liver is not formed.

In more specific embodiments, the subject to be treated is a mammal, e.g., such as cows, pigs, horses, chickens, cats, dogs, etc. In a more specific embodiment, the subject is a human subject in need of such treatment.

In additional embodiments of the methods of the invention, the Tie2 agonist is selected from a group which includes Ang1, Ang2, an anti-Tie2 activating antibody, Ang1*, a fusion protein capable of activating Tie2, for example Ang1FD-FD-Fc, Ang2FD-FD-Fc, Ang1FD-Fc-FD, or Ang2FD-Fc-FD, or a fragment or derivative of Ang1, Ang2, or an anti-Tie2 activating antibody which is capable of activating Tie2. In further embodiments, the Tie2 agonist is a small molecule, lipid, aptamer, nucleic acid, or carbohydrate. Still another embodiment is a method wherein the Tie2 agonist is administered in combination with another agent, for example, VEGF, in particular, VEGF-C or VEGF-D.

Other objects and advantages will become apparent from a review of the ensuing detailed description.

### DETAILED DESCRIPTION OF THE INVENTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to describe the methods and/or materials in connection with which the publications are cited.

### DEFINITIONS

By the term “therapeutically effective dose” is meant a dose that produces the desired effect for which it is administered. The exact dose will depend on the purpose of the treatment, and will be ascertainable by one skilled in the art using known techniques (see, for example, Lloyd (1999) The Art, Science and Technology of Pharmaceutical Compounding).

By the term “activator” is meant a substance that initiates or causes a chemical or physiological reaction or response. Common activators include but are not limited to agonists, activating antibodies, small molecule agonists and derivatives thereof. More specifically, an example of a Tie2 activator is a Tie2 agonist such as Ang1, Ang2, Ang1*, Ang1FD-FD-Fc, Ang2FD-FD-Fc, Ang1FD-Fc-FD, or Ang2FD-Fc-FD. For a complete description of Ang1*, see U.S. Pat. Nos. 6,265,564 and 6,441,137. For a complete description of Ang1FD-FD-Fc, Ang2FD-FD-Fc, Ang1FD-Fc-FD, or Ang2FD-Fc-FD, see International Publication No. WO 00/37642, the contents of which are incorporated herein by reference.

Generation of Antibodies to Activate Tie2 Receptor

The term “antibody” as used herein refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant regions, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Within each IgG class, there are different isotypes (eg. IgG₁, IgG₂, etc.). Typically, the antigen-binding region of an antibody will be the most critical in determining specificity and affinity of binding.

An exemplary immunoglobulin (antibody) structural unit comprises a tetramer. Each tetramer is composed of two identical pairs of polypeptide chains, each pair having one light chain (about 25 kD) and one heavy chain (about 50-70 kD). The N-terminus of each chain defines a variable region of about 100-110 or more amino acids primarily responsible for antigen recognition. The terms “variable light chain” (V_{L}) and variable heavy chain (V_{H}) refer to these light and heavy chains respectively.

Antibodies exist as intact immunoglobulins, or as a number of well-characterized fragments produced by digestion with various peptidases. For example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the terms antibody, as used herein, also includes antibody fragments either produced by the modification of whole antibodies, or those synthesized de novo using recombinant DNA methodologies (e.g., single chain Fv)(scFv) or those identified using phase display libraries (see, for example, McCafferty et al. (1990) Nature 348:552-554).

Methods for preparing antibodies are known to the art. See, for example, Kohler & Milstein (1975) Nature 256:495-497; Harlow & Lane (1988) Antibodies: a Laboratory Manual, Cold Spring Harbor Lab., Cold Spring Harbor, N.Y.). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity. Techniques for the production of single chain antibodies or recombinant antibodies (U.S. Pat. No. 4,946,778; U.S. Pat. No. 4,816,567) can be adapted to produce antibodies used in the fusion proteins and methods of the instant invention. Also, transgenic mice, or other organisms such as other mammals, may be used to express human or humanized antibodies. Alternatively, phage display technology can be used to identify antibodies and heteromeric Fab fragments that specifically bind to selected antigens.

Antibody Screening and Selection

Screening and selection of preferred antibodies can be conducted by a variety of methods known to the art. Initial screening for the presence of monoclonal antibodies specific to a target antigen may be conducted through the use of ELISA-based methods, for example. A secondary screen is preferably conducted to identify and select a desired monoclonal antibody for use in construction of the multi-specific fusion proteins of the invention. Secondary screening may be conducted with any suitable method known to the art. One preferred method, termed “Biosensor Modification-Assisted Profiling” (“BiaMAP”) is described in co-pending U.S. Ser. No. 60/423,017 filed Nov. 1, 2002, herein specifically incorporated by reference in its entirety. BiaMAP allows rapid identification of hybridoma clones producing monoclonal antibodies with desired characteristics. More specifically, monoclonal antibodies are sorted into distinct epitope-related groups based on evaluation of antibody:antigen interactions.

Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Administration may be acute or chronic (i.e. daily, weekly, monthly, etc.). Such administration may be alone or in combination with other agents. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes. In addition, pre-treatment of a tissue or organ with the agent prior to transplant of such tissue or organ may be desirable.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cellsurface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Pharmaceutical Compositions

The present invention also provides pharmaceutical compositions. Such compositions comprise a therapeutically effective amount of an active agent, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention that will be effective in the treatment of lymphatic system defects can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration, and the judgment of the prescribing physician.

The therapy may be repeated intermittently while symptoms are detectable or even when they are not detectable. The therapy may be provided alone or in combination with other drugs.

Specific Embodiments

The profound and generalized lymphatic dysfunction in mice lacking Ang2 correlates with anatomical abnormalities in both the large and small lymphatics of these animals. These mice apparently are defective in forming large lymphatic channels, which instead are replaced by a ragged mesh of lymphatic networks that are poorly associated with surrounding smooth muscle cells. In addition, small lymphatics in mice lacking Ang2 also exhibit obvious patterning abnormalities. It is intriguing that lymphatic tracking and patterning seems to integrally relate to that of associated arterial vessels, and that both the lymphatics and arterial vessels can be sources of Ang2.

Ang2 seems to play a key role in remodeling and maturation of the lymphatics, in a manner that is absolutely required for their normal function, as suggested for Ang1 within the blood vasculature. Because Ang1 is able to rescue the lymphatic defect, Ang2 appears to be acting as an activating agonist in this situation.

### EXAMPLES

### Example 1

Construction of Targeting Vectors and Embryonic Stem Cell Manipulations

The 5′ and 3′ Ang2 gene fragments used in the construction of the targeting vectors were isolated from a 129SV mouse genomic library in the Lambda FixII vector (Stratagene). The 5′ fragment, a 4.3 kb Sac1 fragment, was inserted into a Not1 site (via Not1 linkers) upstream of a cassette containing a promoter-less LacZ or mouse Ang1 cDNAs, and an SV40 polyadenlyation signal followed by the neomycin-resistance gene driven by the phosphoglycerate kinase promoter (PGK-neo). The LacZ gene contained a modified Kozak sequence. The 3′ homology was comprised of a 5.5 kb Xba1 fragment and was blunted and cloned into a blunt Hind3 site between the PGK-neo and thymidine kinase (tk) genes to generate the final targeting vector. The targeting strategy resulted in a 310 bp deletion in the first exon, spanning the translation initiation site and the first 257 bp (85 codons) of the Ang2 coding region, and leaving the LacZ or Ang1 cDNA inserted into the 5′ untranslated region of the first Ang2 exon (53 bp upstream of the natural initiation codon), and thus under control of the native Ang2 promoter. The targeting vectors were linearized at a Srf1 site in the polylinker upstream of the 5′ homology arm, electroporated into E14.1 embryonic stem (ES) cells (derived from 129/Ola mice), and subjected to a double selection scheme utilizing G418 and gancyclovir. Positive ES clones were identified at a rate of 13% by Southern analyses using probes external to the homology arms. Two independent ES cell clones for each line were used to generate chimeric mice using standard injection protocols. Germline-transmitting chimeric mice were bred to C57/BL6 mice, as were F1 progeny. Phenotypes were evaluated through the F9 generation on the C57/BL6 background for LacZ insertions and through F2 with Ang1 insertion.

Northern analysis: Standard Northern analysis was performed. Ang1 and Ang2 probes were derived from full-length mouse cDNAs. Reverse transcription was performed using the Thermoscript RT-PCR system (Invitrogen Carlsbad, Calif.), and PCR was performed using PCR Supermix High Fidelity from (Invitrogen). PCR was performed using forward primers derived from the 5′ UT of the Ang2 message in conjunction with reverse primers from either Ang2 or Ang1 to detect either endogenous mRNA or inserted Ang1 cDNA.

Staining whole mount and thick tissues with LYVE-1 antibody: Intestine, ear skin, and other tissues were collected and put into fix (1% paraformaldehyde in PBS, pH 7.4) for approx 1 hr, and then washed with PBS. Some tissues were processed as whole mounts (e.g., ear skin, intestine), whereas other tissues and parts of the intestine were embedded in warmed (40° C.) low-melting point agarose (FMC) in (7% in PBS). Transverse sections of intestine were cut (100 μm thickness) with a Vibratome. Tissue was blocked (3% goat serum in PBS+0.3% Triton X-100, 2 hrs), then stained with rabbit anti-LYVE-1 (1:2,000) and hamster anti-PECAM (1:500-Serotec), in PBS+0.3% Triton X-100, followed by FITC goat anti-hamster (1:500-Jackson Immunoresearch) plus Cy3 goat anti-rabbit (1:500-Jackson Immunoresearch) in PBS+0.3% Triton X-100. Tissues were mounted in Vectashield (Vector Laboratories) and viewed with a confocal microscope (Leica). Alternatively, to visualize anti-LYVE-1 staining with light microscopy, tissues were fixed, dehydrated through 25%, 50%, 75%, 100% MeOH. Tissues were then bleached in 5% H₂O₂/MeOH (5 hrs), rinsed with 100% MeOH twice, rehydrated through 75%, 50%, 25% MeOH/PBS, blocked in blocking solution (0.5% BSA, 0.1% TX-100 in PBS for 1 hr). Primary antibody was incubated as above in blocking solution overnight at 4° C., washed extensively with PBT (0.2% BSA, 0.1% TX-100 in PBS), incubated in secondary antibody (goat anti-rabbit-biotin, Vector BA-1000, 1:1,500 dilution) in blocking solution overnight at 4° C. Tissues were then washed extensively with PBT, incubated in Avidin:Biotin:Peroxidase complex (Vector, Elite PK-6100, 1:2,000 dilution) in blocking solution overnight at 4° C., washed extensively, equilibrated in DAB developing buffer.

### Example 2

Engineering of Mice Lacking Ang2

To generate mice lacking Ang2, Applicants first constructed a targeting vector that replaced part of the coding region of Ang2 with the LacZ gene encoding β-galactosidase (β-gal), with the intention of creating a null allele that substituted β-gal as a reporter for normal Ang2 expression patterns (referred to as an “Ang2-LacZ knockout allele”, Ang2^{LZ}). This targeting vector was used to alter the endogenous Ang2 allele in embryonic stem cells, which were then used to generate mice carrying this altered allele. Mice homozygous for the altered allele (Ang2^{LZ/LZ}) lacked detectable Ang2 encoding transcripts. Mice heterozygous for this allele (Ang2^{+/LZ}) were overtly normal, and mice homozygous for the altered allele were born at normal frequencies. However, the vast majority (95-99%) of homozygous mice died by two weeks of age. Although overtly normal at birth, the homozygous mice suffered from increasingly obvious and severe chylous ascites and lymphatic dysfunction shortly after feeding (described below).

### Example 3

Validation of LacZ as an Accurate Reporter of Ang2 Expression

To validate the use of the LacZ gene as an accurate reporter for Ang2 gene expression, Applicants first stained newborn mice heterozygous for the Ang2-LacZ allele for β-gal activity. According to this reporter, the major site of Ang2 expression was in smooth muscle cells of large arteries, beginning where the aorta and pulmonary artery exited the heart, and extending into the major arterial branches. Expression was also in large veins and venules, but not in smaller vessels or in the endothelium of quiescent vessels. However, Ang2 expression as assessed by the reporter was induced within the endothelium of small vessels at sites of vascular remodeling (see below), consistent with prior observations using in situ hybridization methods. In contrast, Ang1 expression, as assessed by an analogous LacZ reporter system, was very high within all four chambers of the heart, but absent in the great vessels emanating from the heart. Confirming the faithfulness of the inserted LacZ gene as a reporter for both the Ang1-LacZ and Ang2-LacZ mice, Northern analysis revealed that in normal mice, endogenous Ang1 transcripts are indeed specifically expressed in the heart and not the aorta, while endogenous Ang2 transcripts are found in the aorta and not the heart.

The robust expression of Ang2 by smooth muscle cells in large arterial vessels suggested that it might have a critical function in the walls of these vessels. However, initial structural analyses has not revealed any gross abnormalities in the arteries of young mice lacking Ang2, although it is possible that subtle abnormalities in structure or function might become manifest if detailed studies could be performed in mature mice. However, as noted above, only rare animals survive past two weeks of life, and these animals are severely compromised by generalized lymphatic dysfunction (see below). As will be discussed, the arterial expression of Ang2 could also be consistent with a role not only within the arteries themselves, but in the patterning of lymphatic vessels adjacent to arteries.

### Example 4

Ang2 is Required for Lymphatic Function

As noted above, newborn mice lacking Ang2 developed chylous ascites shortly after birth, upon their initial feedings, which was obvious in more than 90% of the pups by P2. While normal pups had milk only within their digestive tracts, the entire peritoneal cavity of Ang2^{LZ/LZ} pups was filled with a milky fluid, which was also frequently found in the pleural cavity. Milky fluid could uniformly be collected from the peritoneal cavities of Ang2^{LZ/LZ} pups, but not from their normal littermates. Biochemical analysis of this fluid confirmed its chylous character (mean triglyceride level of 2770 mg/dl, in contrast to normal plasma, which has triglyceride levels below 200 mg/dl).

Chylous ascites is characteristic of defective lymphatic function, resulting from poor uptake and transport of chyle produced by the intestine following feeding. Consistent with generalized lymphatic dysfunction, the skin of Ang2^{LZ/LZ} pups was obviously raised and translucent, indicating subcutaneous edema. Rare Ang2^{LZ/LZ} mice surviving to adulthood often exhibited severe clear or chylous ascites, as well as features of chronic lymph stasis throughout the body.

To directly assess lymphatic function in Ang2^{LZ/LZ} pups, uptake and transport of protein-bound Evans blue dye (EBD) by the lymphatics was assessed. In normal 12 day old pups, EBD injected intradermally into the hindlimb promptly stained lymphatic channels in the skin, then regional and more distal lymph nodes, until stain could be seen within retroperitoneal paraaortic lymph nodes and their efferent lymphatics that then emptied into the cisterna chyli and finally into the thoracic duct. In contrast, when EBD was similarly injected into Ang2^{LZ/LZ} pups, the dye spread uniformly away from the injection site, apparently diffusing through the subcutaneous tissue, but was not taken up by lymphatic channels in the skin or elsewhere in any of the 11 Ang2^{LZ/LZ} mice examined.

### Example 5

Larger Lymphatic Channels in Ang2^{LZ/LZ} Pups Display Defects in Structure which Correlate with Lymphatic Dysfunction

Altogether, the chylous ascites, subcutaneous edema, and failed EBD uptake assays, demonstrated severe and generalized lymphatic dysfunction in the Ang2^{LZ/LZ} mice. In an attempt to understand the anatomical basis of this lymphatic dysfunction, the mesenteric vessels in the peritoneal cavity of Ang2^{LZ/LZ} mice were closely examined upon necropsy. As in control littermates, large mesenteric lymphatic vessels were clearly present and filled with chyle in Ang2^{LZ/LZ} pups, and were normally positioned adjacent to mesenteric arteries and veins. Thus, Ang2 is not required for the formation of lymphatics. However, the large lymphatics in the Ang2^{LZ/LZ} pups often had a conspicuously ragged and lacy appearance, compared to the smooth contours of the corresponding lymphatic channels in control pups. In addition, the large mesenteric vessels of the Ang2 knockout pups were surrounded by a diffuse halo of chylous fluid, indicating leakage. Confocal microscopic analysis of fluorescently labeled lymphatics confirmed the abnormal architecture of the large lymphatic vessels in the Ang2^{LZ/LZ} pups. In contrast to the well-organized large lymphatic channels in control pups (FIG. 3G), the lymphatics in corresponding regions of Ang2^{LZ/LZ} pups were often disorganized and formed lacy networks.

Because some of the vascular defects seen in mice lacking Ang1 have been attributed to disrupted interactions between the vascular endothelium and supporting smooth muscle cells, Applicants examined the lymphatics for their smooth muscle investiture. Indeed, whereas the well-defined lymphatic channels seen in control pups were closely enveloped by smooth muscle cells, the disorganized lymphatic networks found in Ang2^{LZ/LZ} mice were often surrounded by poorly associated clusters of smooth muscle cells.

Finally, to determine whether Ang2 was expressed in the vicinity of these abnormal lymphatics, β-gal reporter assays were performed on the mesentery of Ang2-LacZ mice. Reporter expression was clearly noted not only in the large mesenteric arteries, but within the aberrant lymphatics in these mice. In addition, the receptor for Ang2, Tie2, is expressed by cultured lymphatic endothelium.

These findings are consistent with a model in which local Ang2 expression, provided by the lymphatics themselves and/or adjacent large blood vessels, acts on Tie2 receptors within the lymphatics in a manner that is necessary for proper lymphatic development. As noted below, gene rescue studies in which the Ang2 coding region is replaced with that of Ang1 further support the model that Ang2 is acting as an agonist of Tie2 during lymphatic development.

### Example 6

Smaller Lymphatic Vessels in Ang2^{LZ/LZ} Pups Display Abnormal Patterning

The above findings were obtained from the analysis of large-caliber lymphatic channels. To determine whether smaller-sized lymphatics also had abnormalities in Ang2^{LZ/LZ} pups, Applicants first examined small-caliber intestinal lymphatics involved in the uptake and transport of chyle. Whole-mount staining of intestines with the lymphatic-specific LYVE-1 antibody revealed dramatic differences in the patterning of intestinal lymphatics in Ang2^{LZ/LZ} pups compared to their control littermates. While intestinal lymphatics were dense and well-organized in control pups, they were disorganized and irregular in the Ang2^{LZ/LZ} pups. In control pups, the dense lymphatics typically formed regular patterns surrounding the major blood vessels; this regular patterning of lymphatics around arteries suggests a relationship between the guidance and patterning of lymphatics and arteries, and may relate to their shared expression of Ang2. In contrast, the intestinal lymphatics in the Ang2^{LZ/LZ} pups did not display these regular patterns, and instead contained regions largely bereft of lymphatics with regions containing sparse and disorganized lymphatics.

Cross-sections of intestines, simultaneously labeled for both blood vessels and lymphatics also revealed dramatic abnormalities in the Ang2^{LZ/LZ} pups. In the intestinal villi of control pups, the central lymphatic lacteal, which is the major site of lipid uptake in the intestine, was surrounded by a basket of blood vessels. In contrast, the central lacteals of Ang2^{LZ/LZ} pups were frequently absent, and when present were abnormally short, although the villus blood vessels appeared normal. In addition, examination of the cross-sections from the Ang2^{LZ/LZ} pups revealed that their lymphatics were generally sparser and much more dilated than those in control pups.

Consistent with the generalized lymphatic dysfunction seen in Ang2^{LZ/LZ} mice, obvious lymphatic patterning differences reminiscent of those in the gut are also noted in the skin of Ang2^{LZ/LZ} pups as compared to their normal littermates.

### Example 7

Rescue of Lymphatic Defects, but not Blood Vessel Remodeling Defects, Following Gene Replacement with Ang1

Because in vitro studies indicate that Ang2 can act as either an activator (agonist) or blocker (antagonist) of its Tie2 receptor, depending on the cell type and context, it remained unclear as to whether the above defects were due to loss of Ang2 agonistic or antagonistic activities. To gain insight into this issue, Applicants performed a genetic swap, replacing the Ang2 gene with a cDNA encoding Ang1 (an obligate agonist of Tie2), using a targeting scheme analogous to that used to replace Ang2 with lacZ. Maybe something brief should be said about targeting scheme? After targeting in ES cells and generation of mice heterozygous for the Ang2 allele (designated Ang2^{A1/+}), matings generated mice in which both alleles of Ang2 were replaced with Ang1 (Ang2^{A1/A1}) as confirmed by Southern blotting. RT-PCR analysis revealed that whereas endogenous Ang2 transcripts could no longer be detected in these mice, a unique fusion transcript specific for the introduced Ang1 cDNA was now expressed in the organs that normally expressed Ang2.

The replacement of the Ang2 gene with Ang1 appeared to entirely rescue the lymphatic defects found in Ang2^{LZ/LZ} mice. Ang2^{A1/A1} pups appeared healthy and did not display chylous ascites. The patterning of the lymphatic vessels in the intestinal wall of Ang2^{A1/A1} pups was indistinguishable from control mice and revealed none of the defects of Ang2^{LZ/LZ} mice, showing normal organization of lymphatic vessels around the feeding arterioles. In addition, the villi of Ang2^{A1/A1} pups contained central lacteals of normal length and number. Thus, Ang1 can replace Ang2 in guiding the normal development and patterning of lymphatic vessels.

In contrast to the rescue of the lymphatic defects, replacement of the Ang2 gene with the Ang1 gene did not rescue the defects in vascular remodeling in the neonatal eye. The hyaloid vessels of Ang2^{A1/A1} pups persisted to at least P10, similar to those in Ang2^{LZ/LZ} mice. Furthermore, the retinal vessels failed to grow normally to the retinal periphery. Thus, Ang1, an obligate agonist of the Tie2 receptor, can completely substitute for Ang2 during lymphatic development but not during vascular remodeling in the postnatal eye.

We claim:

1. A method of promoting functional lymphatic vessel formation in a mammal comprising administering to the mammal a Tie2 agonist such that functional lymphatic formation occurs.

2. The method of claim 1, wherein the Tie2 agonist is selected from the group consisting of Ang1, Ang2, anti-Tie2 activating antibody, Ang1*, Ang1FD-FD-Fc, Ang2FD-FD-Fc, Ang1FD-Fc-FD, or Ang2FD-Fc-FD, or a fragment or derivative thereof.

3. The method of claim 1, wherein the Tie2 agonist is a small molecule, lipid, aptamer, nucleic acid, or carbohydrate.

4. The method of claim 1, wherein the Tie2 agonist is administered in combination with VEGF.

5. The method of claim 4 wherein the VEGF is VEGF-C or VEGF-D.

6. The method of claim 1, wherein the administration is subcutaneous, intramuscular, intradermal, intraperitoneal, intravenous, intranasal, or oral routes of administration.

7. A method of inducing lymphatic vessel maturation in a mammal comprising administering to the mammal a Tie2 agonist such that functional lymphatic maturation occurs.

8. The method of claim 7, wherein the Tie2 agonist is selected from the group consisting of Ang1, Ang2, anti-Tie2 activating antibody, Ang1*, Ang1FD-FD-Fc, Ang2FD-FD-Fc, Ang1FD-Fc-FD, or Ang2FD-Fc-FD, or a fragment or derivative thereof.

9. The method of claim 7, wherein the Tie2 agonist is a small molecule, lipid, aptamer, nucleic acid, or carbohydrate.

10. The method of claim 7, wherein the Tie2 agonist is administered in combination with VEGF.

11. The method of claim 10 wherein the VEGF is VEGF-C or VEGF-D.

12. The method of claim 7, wherein the administration is subcutaneous, intramuscular, intradermal, intraperitoneal, intravenous, intranasal, or oral routes of administration.

13. A method of preventing chylous ascites formation in a mammal comprising administering to the mammal a Tie2 agonist such that chylous ascites is not formed.

14. The method of claim 13, wherein the Tie2 agonist is selected from the group consisting of Ang1, Ang2, anti-Tie2 activating antibody, Ang1*, Ang1FD-FD-Fc, Ang2FD-FD-Fc, Ang1FD-Fc-FD, or Ang2FD-Fc-FD, or a fragment or derivative thereof.

15. The method of claim 13, wherein the Tie2 agonist is a small molecule, lipid, aptamer, nucleic acid, or carbohydrate.

16. The method of claim 13, wherein the Tie2 agonist is administered in combination with VEGF.

17. The method of claim 16 wherein the VEGF is VEGF-C or VEGF-D.

18. The method of claim 13, wherein the administration is subcutaneous, intramuscular, intradermal, intraperitoneal, intravenous, intranasal, or oral routes of administration.

19. A method of treating lymphedema in a mammal comprising administering to the mammal Tie2 agonist such that lymphedema is treated.

20. The method of claim 19, wherein the Tie2 agonist is selected from the group consisting of Ang1, Ang2, anti-Tie2 activating antibody, Ang1*, Ang1FD-FD-Fc, Ang2FD-FD-Fc, Ang1FD-Fc-FD, or Ang2FD-Fc-FD, or a fragment or derivative thereof.

21. The method of claim 19, wherein the Tie2 agonist is a small molecule, lipid, aptamer, nucleic acid, or carbohydrate.

22. The method of claim 19, wherein the Tie2 agonist is administered in combination with VEGF.

23. The method of claim 22 wherein the VEGF is VEGF-C or VEGF-D.

24. The method of claim 19 wherein the administration is subcutaneous, intramuscular, intradermal, intraperitoneal, intravenous, intranasal, or oral routes of administration.

25. A method of decreasing ascites associated with cirrhosis of the liver in a mammal comprising administering to a Tie2 agonist to the mammal such that ascites associated with cirrhosis of the liver is decreased.

26. The method of claim 25, wherein the Tie2 agonist is selected from the group consisting of Ang1, Ang2, anti-Tie2 activating antibody, Ang1*, Ang1FD-FD-Fc, Ang2FD-FD-Fc, Ang1FD-Fc-FD, or Ang2FD-Fc-FD, or a fragment or derivative thereof.

27. The method of claim 25, wherein the Tie2 agonist is a small molecule, lipid, aptamer, nucleic acid, or carbohydrate.

28. The method of claim 25, wherein the Tie2 agonist is administered in combination with VEGF.

29. The method of claim 28 wherein the VEGF is VEGF-C or VEGF-D.

30. The method of claims 25, wherein the administration is subcutaneous, intramuscular, intradermal, intraperitoneal, intravenous, intranasal, or oral routes of administration.
